(19) United States
(12) Patent Application Publication
(10) Pub. No.: US 2007/0287158 A1
(43) Pub. Date: Dec. 13, 2007

(54) Cell collection and disease screening

(76) Inventors: George Gorodeski, Beachwood, OH (US); Richard Domanik, Libertyville, IL (US)

Correspondence Address: CALFEE HALTER & GRISWOLD, LLP, 800 SUPERIOR AVENUE, SUITE 1400, CLEVELAND, OH, 44114, US

(21) Appl. No.: 11/810,916

(22) Filed: Jun. 7, 2007

Related U.S. Application Data
(60) Provisional application No. 60/812,419, filed on Jun. 9, 2006.

(51) Int. Cl. C12Q 1/68; G01N 33/574; A61M 31/00

(52) U.S. Cl. 435/6; 435/7.23; 604/500

(57) ABSTRACT

The present application discloses methods and tools for collecting cells to screen for disease, such as cancer. In one method of screening for a disease a flush fluid is injected into a human cavity, such as a uterus. The flush fluid and entrained cells from the cavity are withdrawn from the cavity and collected. The intermixed cells may be separated from the fluid and analyzed to determine whether the cells have a characteristic that is indicative of the presence of a disease. One tool for applying and withdrawing a fluid includes a nozzle, a screening fluid supply and fluid withdrawal mechanism. The nozzle includes an inlet port and an outlet port. The screening fluid supply supplies a screening fluid through the outlet port. The withdrawal mechanism withdraws fluid through the outlet port.

### RELATED APPLICATIONS

This application claims the benefit of U.S. provisional patent application Ser. No. 60/812,419 for CELL COLLECTION AND DISEASE SCREENING filed Jun. 9, 2006, the entire disclosure of which is fully incorporated herein by reference.

### FIELD OF THE INVENTION

The present application relates to non-invasive techniques and tools for collecting cells from a body cavity.

### BACKGROUND

Current methods of screening for endometrial cancer are invasive and sample only isolated areas of the endometrium. Current methods of screening for endometrial cancer are invasive, because tissue samples used for the screening are obtained by tearing a small amount of tissue from the endometrial wall. Because the current methods are invasive, screening for endometrial cancer is typically performed only after the patient has developed some symptom that causes endometrial cancer to be suspected. One current method of screening for endometrial cancer involves taking biopsies of endometrial tissue with a cannula. The cannula is composed of an external plastic tubing with an opening at its side near the tip. The tube houses a rod which fits tightly into the external tubing. Before the cannula is inserted into the uterine cavity, the rod is advanced to a closed tip of the external tubing. The cannula is then placed in the uterine cavity, and the rod is pulled away from the tip of the plastic tubing. The movement of the rod creates a negative pressure that draws endometrial tissue into the cannula through a side opening near the tip. A small amount of tissue is sucked into the tubing by the vacuum created by pulling the rod to thereby take a biopsy of the tissue. The cannula samples a tissue fragment obtained from one site of the cavity, at the area where the cannula came in direct contact with the surface lining. The tissue fragment may include stromal cells as well as epithelial cells.

### SUMMARY

The present application discloses methods and tools for collecting cells to screen for disease, such as cancer. In one method of screening for a disease, a flush fluid is injected into a human cavity, such as a uterus. The flush fluid and entrained cells from the cavity are withdrawn from the cavity and collected. The intermixed cells may be separated from the fluid and analyzed to determine whether the cells have a characteristic that is indicative of the presence of a disease.

One tool for applying and withdrawing a fluid includes a nozzle, a screening fluid supply and fluid withdrawal mechanism. The nozzle includes an inlet port and an outlet port. The screening fluid supply supplies a screening fluid through the outlet port. The withdrawal mechanism withdraws fluid through the outlet port.

### BRIEF DESCRIPTION OF THE FIGURES

FIG. 1A is a flow chart of a method of retrieving cells for a cancer screening;

FIG. 1B is a flow chart of a method of screening for cancer;

FIG. 2 is a schematic illustration of a tool for collecting cells disposed in a human body cavity;

FIG. 3 is a schematic illustration of the tool shown in FIG. 2 injecting a screening fluid into the cavity;

FIG. 4 is a schematic illustration of the tool shown in FIG. 2 withdrawing flush fluid from the cavity;

FIG. 5 is a flow chart of a method of retrieving cells from a uterus for a cancer screening;

FIG. 6 is a schematic illustration of a tool for collecting endometrial cells disposed in a uterus;

FIG. 7 is a schematic illustration of the tool shown in FIG. 6 injecting a screening fluid into the uterus;

FIG. 8 is a schematic illustration of the tool shown in FIG. 8 withdrawing flush fluid from the uterus;

FIG. 9A is a schematic illustration of a tool for injecting and withdrawing a screening fluid into a human body cavity;

FIG. 9B illustrates the tool shown in FIG. 9B being operated to inject fluid;

FIG. 9C illustrates the tool shown in FIG. 9C being operated to withdraw fluid;

FIG. 10A is a schematic illustration of a tool for injecting and withdrawing a screening fluid into a human body cavity;

FIG. 10B illustrates the tool shown in FIG. 9B being operated to inject fluid;

FIG. 10C illustrates the tool shown in FIG. 9C being operated to withdraw fluid;

FIG. 11 illustrates an arrangement of an inlet nozzle and an outlet nozzle of a tool for injecting and withdrawing fluid;

FIG. 12A illustrates a fluid collection arrangement for use with a tool for injecting and withdrawing fluid;

FIG. 12B is a view taken along lines 12B-12B in FIG. 12A;

FIG. 13A is a schematic illustration of a second type of tool for collecting endometrial cells;

FIG. 13B illustrates the tool of FIG. 13A with a head portion extended;

FIG. 13C illustrates the tool of FIG. 13A with a head portion extended and expanded;

FIG. 13D illustrates the tool of FIG. 13A with a head portion extended and expanded and a cover portion expanded;

FIG. 13E illustrates retraction of the head portion and the cover portion back into the tool;

FIG. 13F illustrates the head portion and the cover portion completely retracted into the tool;

FIG. 13G illustrates extending of the head portion of the tool for cell removal;

FIG. 13H illustrates dipping of the head portion of the tool in a solution for cell removal;

FIG. 14 is a schematic illustration of the tool shown in FIG. 13A disposed in a uterus;

FIG. 15 is a schematic illustration of the tool shown in FIG. 14 applying a collector head in the uterus;

FIG. 16 is a schematic illustration of the tool shown in FIG. 14 moving the collector head in the uterus;

FIG. 17 is a schematic illustration of the tool shown in FIG. 14 extending a collector head cover to facilitate removal of the collector head;

FIG. 18 is a schematic illustration of the tool shown in FIG. 14 withdrawing the collector head into the collector head cover;

FIG. 19 is a schematic illustration of the tool shown in FIG. 14 withdrawing the collector head cover; and

FIG. 20 is a schematic illustration of the tool shown in FIG. 14 being withdrawn from the uterus.

### DETAILED DESCRIPTION OF THE INVENTION

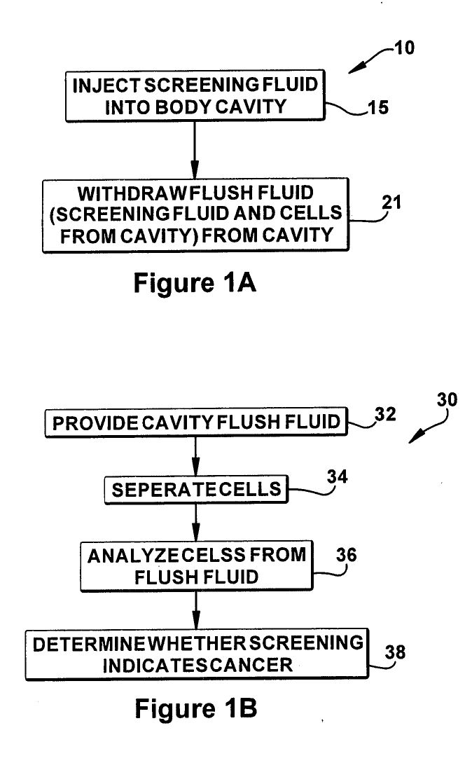

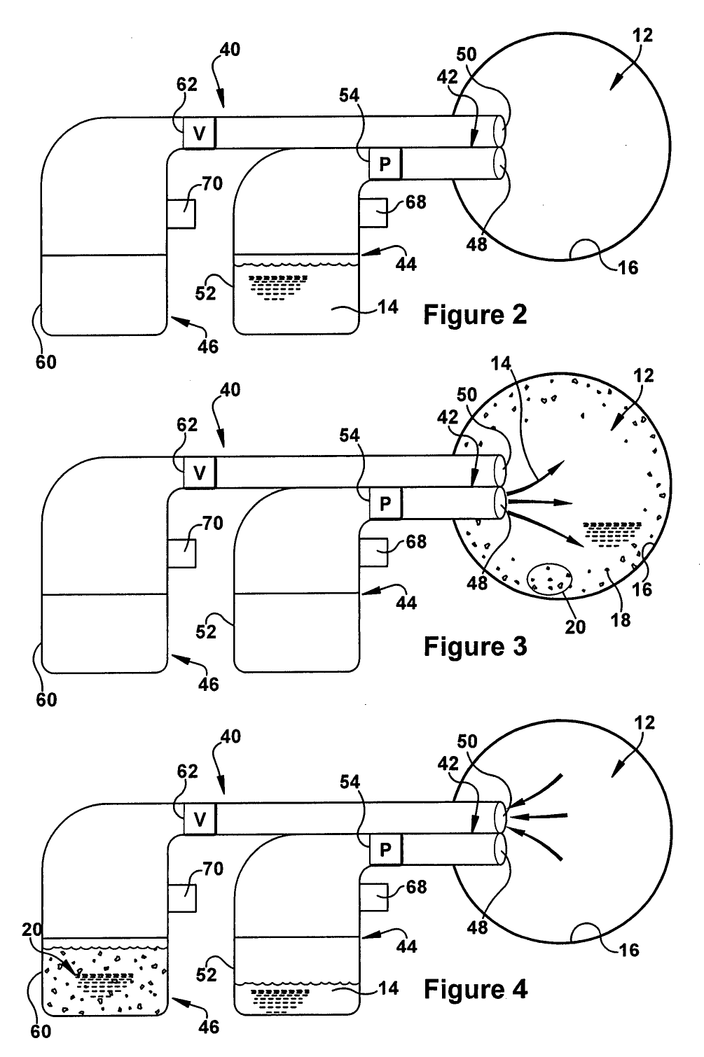

The present application concerns non-invasive methods of collecting cells from a body cavity that can be used to screen for diseases, such as cancer. The screening may be performed to detect a characteristic that is indicative of the presence of cancer cells and/or the presence of pre-cancerous cells. FIGS. 1A and 2-4 illustrate an embodiment of a non-invasive method 10 for collecting cells from a human body cavity 12 (see FIG. 2). In the method, a screening fluid 14 is injected 15 into the body cavity (see FIG. 3). A wide variety of different screening fluids 14 can be used. For example, any fluid that is tolerated by the human body when introduced into the cavity 12 can be used. For example, the screening fluid 14 may comprise saline solution. The screening fluid 14 contacts the lining 16 of the body cavity 12 and cells 18 of the lining of the body cavity become entrained in the screening fluid 14 to form a flush fluid 20. Referring to FIG. 4, the flush fluid 20 is withdrawn 21 from the body cavity.

In one embodiment, the flush fluid 20 from the body cavity is used to screen for cancer and/or the presence of precancerous cells. FIG. 1B illustrates a method 30 of screening for cancer. In the method, the flush fluid with intermixed cells from a human tissue lining of a cavity is provided 32. Optionally the intermixed cells are separated 34 from the flush fluid. The cells from the flush fluid are analyzed 36 to determine 38 whether a characteristic of the cells is indicative of cancer cells and/or precancerous cells in the tissue lining.

The screening fluid 14 can be applied to the cavity by a wide variety of different tools. Any tool that can inject a fluid and then withdraw the fluid can be used. In the example illustrated by FIG. 2, a tool 40 for injecting the screening fluid 14 and withdrawing the flush fluid 20 includes one or more nozzles 42, a screening fluid supply 44, and a flush fluid recovery mechanism 46. The nozzle 42 or nozzles are configured for insertion into the body cavity 12. One nozzle may be included for injecting the screening fluid 14 and a second nozzle may be included for recovering the flush fluid 20. In one embodiment, a single nozzle with a single port is employed. In the example illustrated by FIG. 2, a single nozzle is included that includes a first port 48 for supplying the screening fluid and second port 50 for withdrawing the flush fluid 20. The screening fluid supply 44 supplies screening fluid 14 to the nozzle 42. The screening fluid supply 44 may be any arrangement that is capable of providing screening fluid to the nozzle. The illustrated screening fluid supply 44 includes a screening fluid reservoir 52 and a delivery mechanism 54, such as a mechanical or power operated pump, a plunger arrangement, a collapsible bladder or bulb, etc. The delivery mechanism 54 supplies screening fluid 14 from the reservoir to the cavity 12 through the nozzle 42. In the example illustrated by FIG. 2, the fluid delivery mechanism 54 includes a trigger 68 for selectively actuating the delivery mechanism 54.

The flush fluid recovery mechanism 46 withdraws the flush fluid 20 from the cavity through the nozzle 42. The flush fluid recovery mechanism may be any arrangement that is capable of withdrawing the flush fluid 20 through the nozzle. The illustrated flush fluid recovery mechanism 46 includes a flush fluid reservoir 60 and a fluid removal mechanism 62, such as a manual or power operated vacuum, a plunger arrangement, a collapsible bladder, etc. In one embodiment, the delivery mechanism 54 and the fluid removal mechanism 60 are incorporated in a single device, such as a pump that can be operated to move fluid in two directions. In the exemplary embodiment, the flush fluid reservoir 60 and the screening fluid reservoir 52 are separate receptacles. In an alternate embodiment, when only one injection of fluid is administered and withdrawn to take one sample, the tool may include a single nozzle and a single receptacle and a single mechanism, such as a pump may be operated to both inject and remove the fluid. The fluid removal mechanism 62 removes flush fluid 20 from the cavity 12 through the nozzle 42. In the example illustrated by FIG. 2, the fluid removal mechanism 62 includes a trigger 70 for selectively actuating the fluid removal mechanism 62. In one embodiment, a single trigger replaces the delivery mechanism trigger 68 and the withdrawal mechanism trigger 70. When the single trigger is activated, the tool automatically injects the screening fluid 14 and then withdraws the flush fluid 20.

Referring to FIG. 2, in use, the nozzle or nozzles 42 are inserted into a human body cavity 12. Referring to FIG. 3, once the nozzle 42 is inserted into the cavity, the trigger 68 is activated to inject the screening fluid 14 into the cavity 12. In one embodiment, the screening fluid 14 is injected under a pressure and/or with a velocity that is sufficient to dislodge some cells 18 from the lining 16 of the cavity. When the cavity is a uterus, any pressure can be used that results in sufficient fluid flow velocity at the nuzzle that causes fluid impinging on the uterine wall to displace cells from the surface. A variety of pressure and velocity combinations may be used that are both comfortable for the patient and that are sufficient to dislodge or displace endometrial cells. Cells 18 become entrained in the screening fluid 14 to form the flush fluid 20. The trigger 68 is then deactivated. Referring to FIG. 4, the trigger 70 is then activated to withdraw the flush fluid 20 with entrained cells 18 from the cavity. The trigger is then deactivated. In one exemplary embodiment, the injection and withdraw actions are separate. If the two actions occur simultaneously, it is possible that fluid supplied to the injection port could flow directly to the withdrawal port without contacting the endometrium. In one exemplary embodiment, withdrawal suction and the withdrawal nozzle are configured such that the endometrium is not drawn into the withdrawal nozzle. Drawing the endometrium into the withdrawal nozzle could plug the withdrawal nozzle, thus preventing a complete sample from being collected. If the withdrawal suction applied is too high, larger pieces or chunks of tissue could be pulled into the withdrawal nozzle and also prevent a complete sample from being collected.

This process can be repeated multiple times until an adequate sample of cells is obtained. Further, the cavity 12 may contain debris, such as blood or other matter. Fluid may be injected and withdrawn as many times as is required to cleanse the cavity and remove the debris. Once the debris is removed, a clean flush fluid reservoir 60 can be assembled with the tool. The tool can then be operated to obtain a sample or samples of cells 18 of the cavity lining.

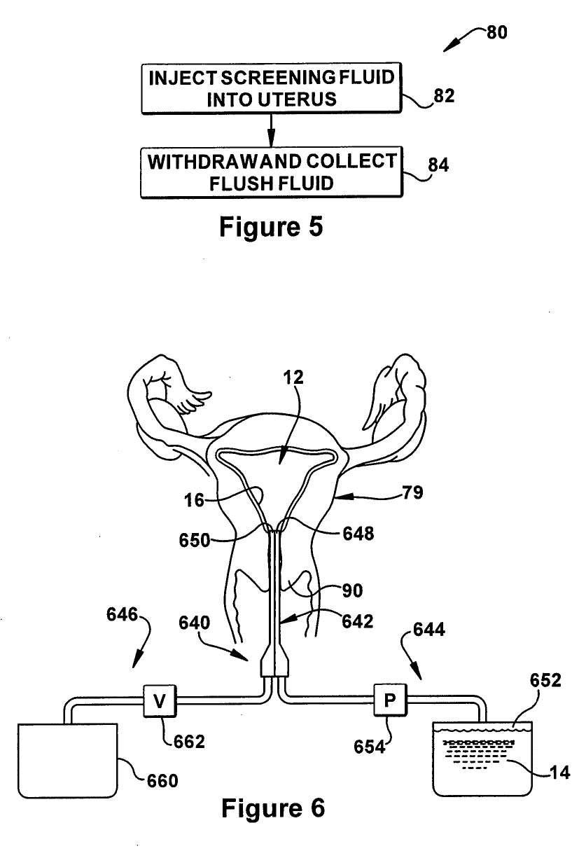

The cavity 12 can be any body cavity where cancer can occur. The cavity may be accessed through an existing orifice of the body or surgically. In one embodiment, the cavity 12 is a human uterus 79 (FIG. 6). FIG. 5 is a flow chart that illustrates a method 80 of obtaining endometrial cells from a human uterus 79 for a cancer screening. In the method, a screening fluid 14 is injected 82 into the uterus. Endometrial cells, such as epithelial endometrial cells, become entrained in the screening fluid 14 to form a flush fluid 20. The flush fluid 20 with entrained endometrial cells is then withdrawn 84 from the uterus and collected.

The flush fluid 20 with entrained endometrial cells, such as epithelial endometrial cells may be analyzed to determine whether the endometrial cells have characteristics that are indicative of cancer of the endometrium. The cells may be analyzed for a wide variety of different characteristics that may be indicative of cancer. For example, the endometrial cells or if the cells are disrupted during processing steps, matter that formed the sample of endometrial cells may be analyzed to determine the presence, absence, ratio, and/or amount of a biomarker in the sample. The presence, absence, ratio, and/or amount of a biomarker in the endometrial cell sample can be determined using one of a wide variety of well know methods. For example, if the biomarker is a protein, antibodies may be used to detect the presence, absence, ratio, and/or amount of the biomarker in the sample. Examples of known methods that use antibodies to detect proteins include imuno-staining, imuno-fluorescence, and western blotting. Biomarkers may also be analyzed in the sample using known messenger RNA detection methods. In one embodiment, the flush fluid 20 is centrifuged to separate the endometrial cells from the fluid to facilitate biomarker analysis of the endometrial cells. The endometrial cells may also be separated using other methods.

In one embodiment, a sample of epithelial endometrial cells are analyzed to determine a condition of a P2X7 biomarker to screen for uterine epithelial cancer. For example, the sample of epithelial endometrial cells may be analyzed for conditions such as the presence, absence, ratio, and/or amount of a P2X7 biomarker in the sample. A P2X7 biomarker system is disclosed in the following publications, which are incorporated herein by reference in their entirety:
Feng Y H, Li X, Wang L, Zhou L, Gorodeski G. A Truncated P2X₇ Receptor Variant (P2X₇₋ⱼ) Endogenously Expressed in Cervical Cancer Cells Antagonizes the Full-Length P2X₇ Receptor through Hetero-Oligomerization. Purinergic Signaling 2:84-85, 2006.
Li X, Zhou L, Feng Y H, Gorodeski G. Biological Significance of the Endogenously Expressed Truncated P2X₇ Receptor Variant (P2X₇₋ⱼ) in Human Uterine and Skin Cancers. Purinergic Signaling 2:132-133, 2006.
Feng Y H, Li X, Wang L, Zhou L, Gorodeski G I. A truncated P2X₇ Receptor Variant (P2X₇₋ⱼ) Endogenously Expressed in Cervical Cancer Cells Antagonizes the Full-Length P2X₇ Receptor Through Hetero-Oligomerization. J Biol Chem (In-Press; Epud ahead of print http://www.jbc.org/cgi/reprint/M602999200v1).

FIG. 6 schematically illustrates an embodiment of a tool 640 configured for insertion into a cervix 90 for injecting a screening fluid 14 and withdrawing a flush fluid 20 from a human uterus 79. The tool 640 includes a slender, elongated nozzle 642 sized for insertion into the uterus 79 of a human patient. The tools 640 includes a screening fluid supply 644, and a flush fluid recovery mechanism 646. The nozzle 642 includes a first port 648 for supplying the screening fluid 14 and second port 650 for withdrawing the flush fluid 20. The screening fluid supply 644 supplies screening fluid 14 to the nozzle 42. The screening fluid supply 644 includes a screening fluid reservoir 652 and a delivery mechanism 654, such as a mechanical or power operated pump. The delivery mechanism 654 supplies screening fluid 14 from the reservoir to the uterus 79 through the nozzle 642.

The flush fluid recovery mechanism 646 withdraws the flush fluid 20 from the uterus 79 through the nozzle 642. The flush fluid recovery mechanism 646 includes a flush fluid reservoir 660 and a fluid removal mechanism 662, such as a manual or power operated vacuum. In the exemplary embodiment, the flush fluid reservoir 660 and the screening fluid reservoir 652 are separate receptacles. The fluid removal mechanism 662 removes flush fluid 20 from the uterus 79 through the nozzle 642.

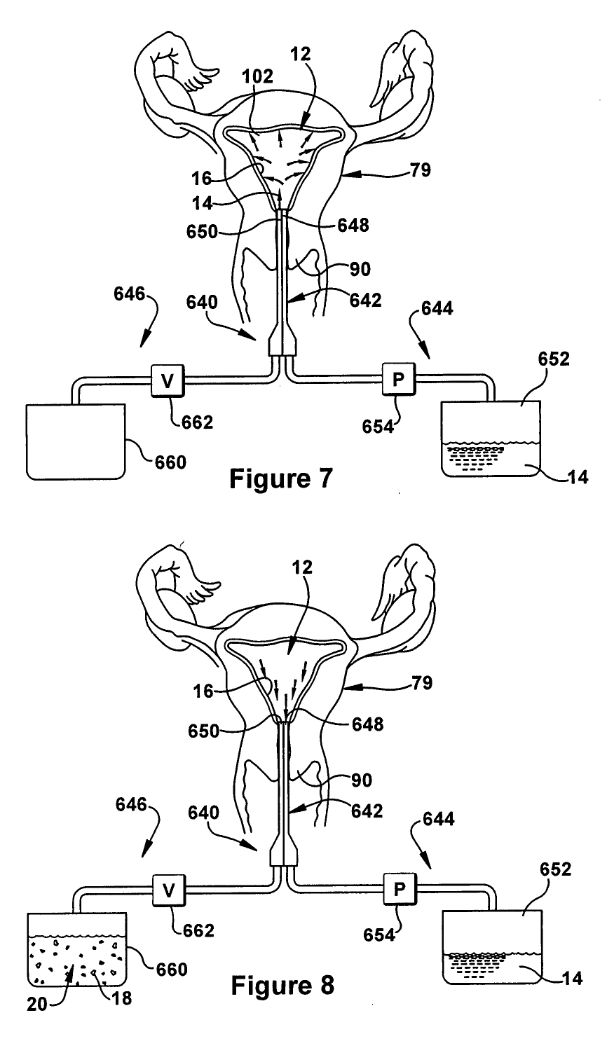

Referring to FIG. 6, in use, the nozzle 642 is inserted into the uterus 79. Referring to FIG. 7, once the nozzle 642 is inserted into the uterus, the screening fluid 14 is injected into the uterus 79. In one embodiment, the screening fluid 14 is injected under pressure that is sufficient to dislodge some epithelial endometrial cells 102. Endometrial cells 102, such as epithelial endometrial cells, become entrained in the screening fluid 14 to form flush fluid 20 (shown in the reservoir 660 in FIG. 8). The flush fluid 20 with entrained cells 18 is withdrawn from the uterus 79. This process can be repeated multiple times until an adequate sample of cells is obtained. Further, the uterus 79 may contain debris, such as blood or other matter. Fluid may be injected and withdrawn as many times as is required to cleanse the uterus and remove the debris. Once the debris is removed, a clean flush fluid reservoir 660 can be assembled with the tool, without removing the tool from the uterus. The tool 640 can then be operated to obtain a sample or samples of epithelial endometrial cells. The tool can then be removed from the uterus.

The endometrial cell collection method and tool allows sampling of most, if not all of the uterine surface and is therefore much more likely to detect a condition that is indicative of cancer than very localized biopsies. Injecting and removing fluid into the uterus is far less invasive than taking a biopsy. Because the disclosed endometrial cell collection method is non-invasive, the method can be implemented to screen for cancer before symptoms develop and thereby detect cancer much earlier. In the endometrium the P2X7 (mRNA and protein) is expressed primarily by the epithelial cells that form the surface lining of the tissue. In the exemplary embodiment, the injection and removal of fluid into the uterus samples mostly epithelial cells and a very small (if any) stromal component. By collecting predominantly epithelial cells the accuracy of cancer detection can be expected to increase when compared with biopsies that may collect stromal as well as endometrial cells.

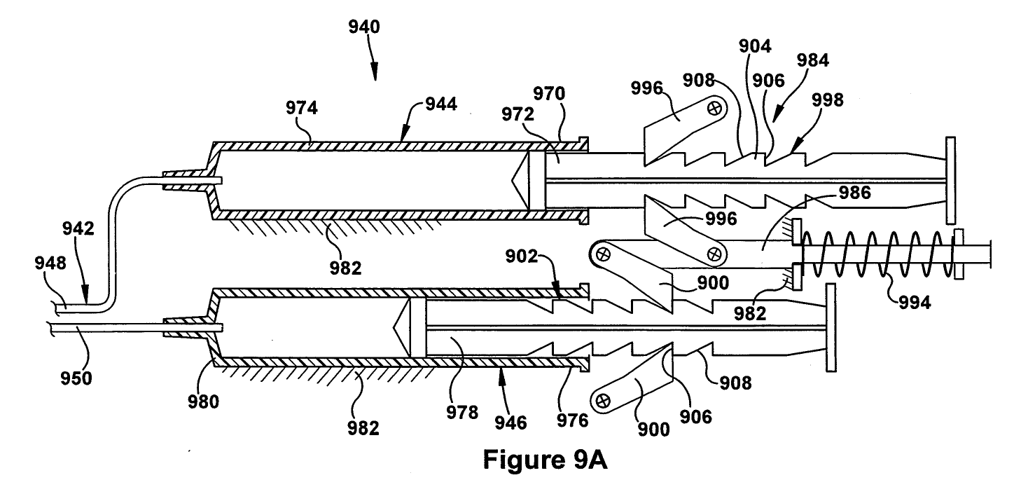

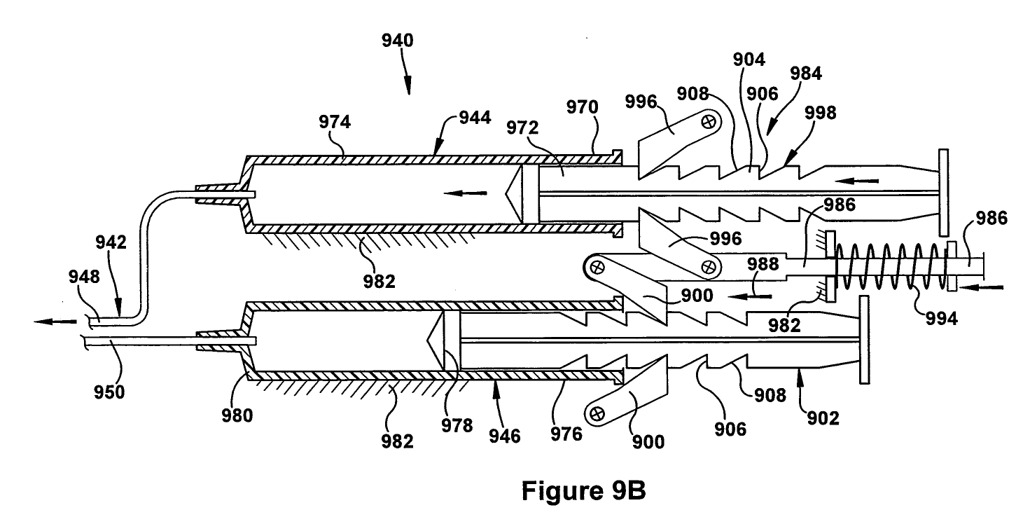

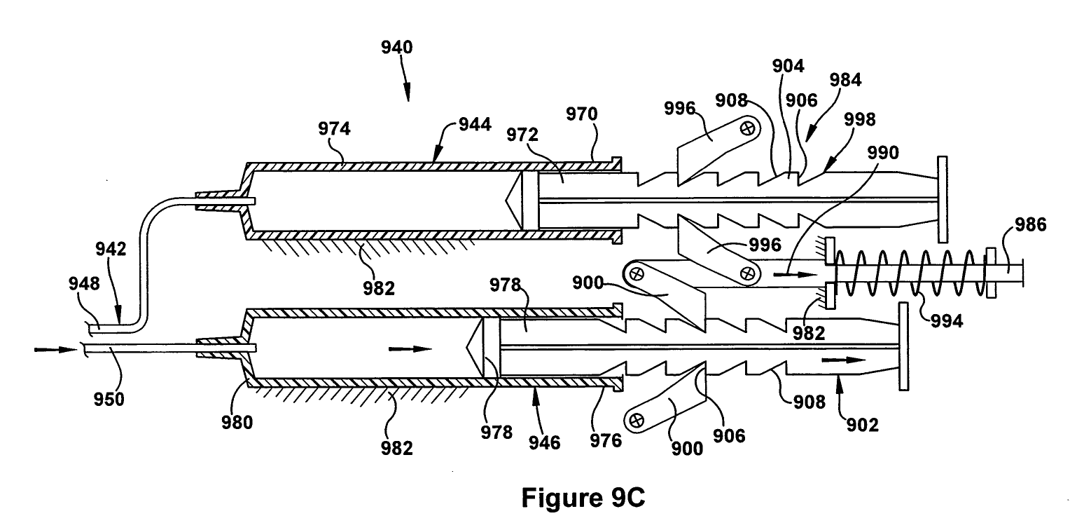

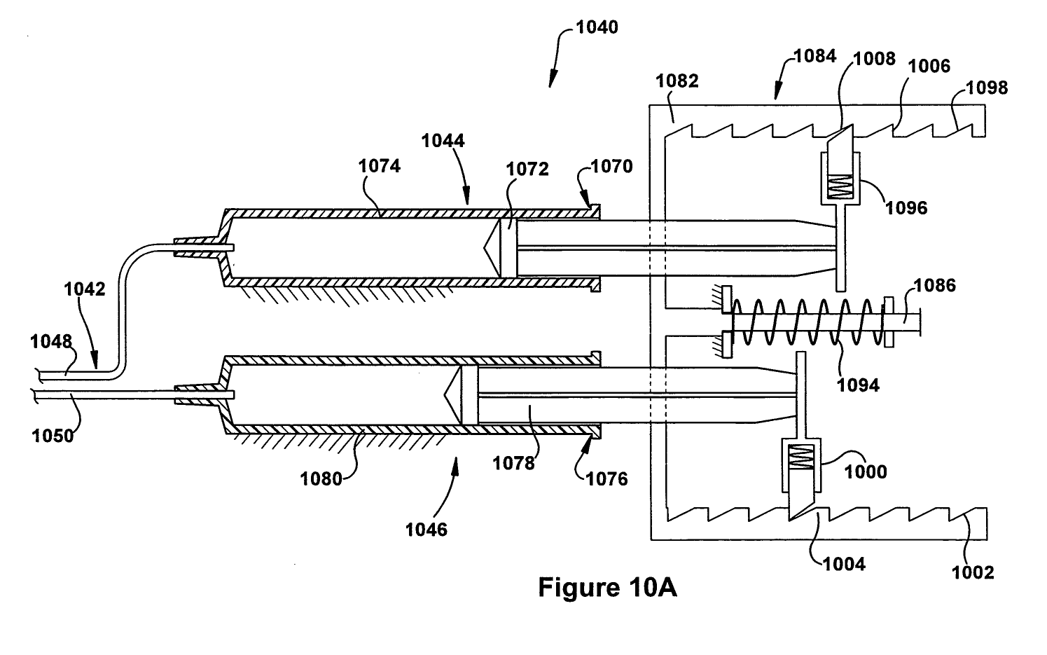

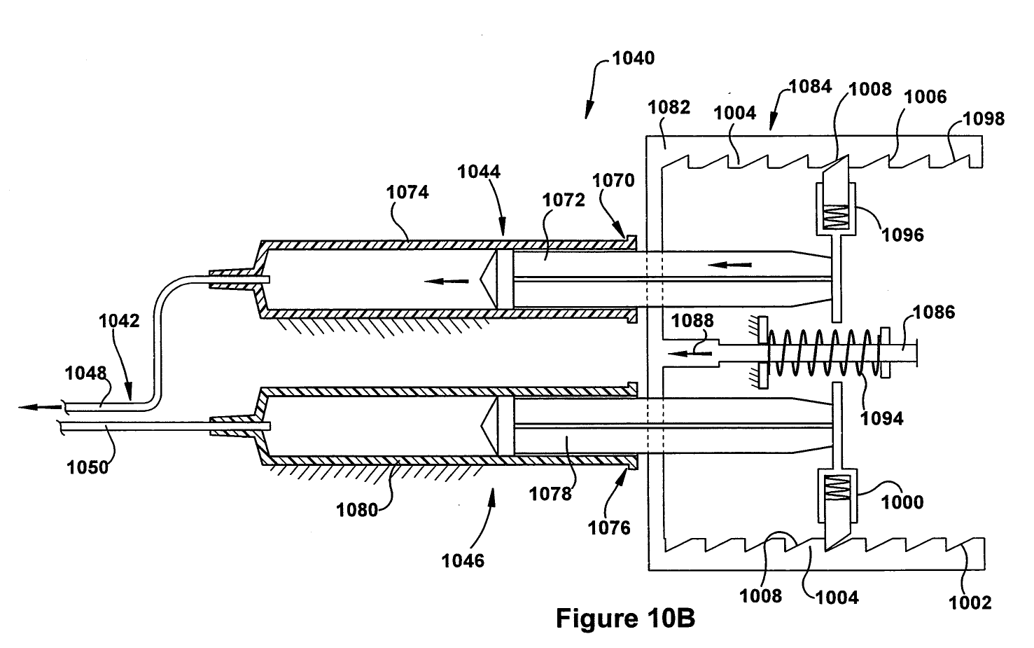

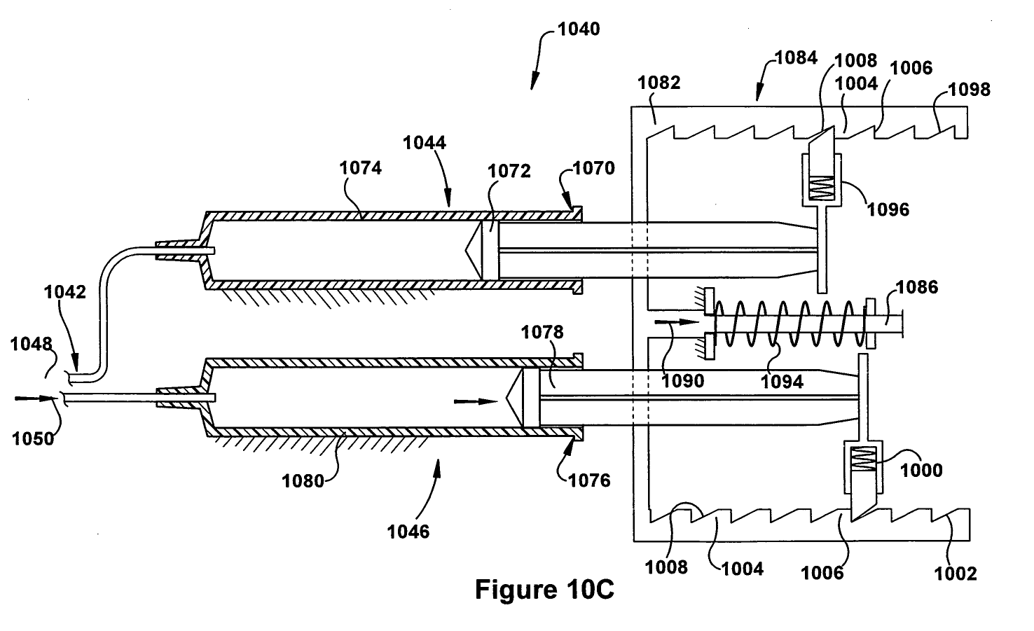

The tools 40, 640 can take a wide variety of different forms. FIGS. 9A and 10A illustrate two examples of the wide variety of different configurations of tools that can be used to inject and withdraw fluid from a body cavity. FIG. 9A illustrates a tool 940 that includes a nozzle 942 with an outlet port 948 and an inlet port 950. The nozzle 942 may take a wide variety of different forms. For example, the nozzle 942 may comprise two elongated tubes as shown in FIG. 9A. The tool 940 includes a screening fluid supply 944 and a flush fluid recovery mechanism 946. In the exemplary tool 940 illustrated by FIG. 9A, the screening fluid supply 944 comprises a syringe 970 that includes a plunger 972 and a reservoir or body 974. The flush fluid recovery mechanism 946 comprises a syringe 976 that includes a plunger 978 and a reservoir or body 980. The syringe bodies 974, 980 are fixed to a frame member indicated schematically by reference number 982. The tool 940 also includes a ratchet mechanism 984 that is coupled to the screening fluid syringe 970 and the withdrawal syringe 976.

Referring to FIG. 9B, when an operating member 986 of the ratchet mechanism is moved in a direction illustrated by arrow 988, the plunger 972 of the screening fluid syringe 970 is forced into the body 974, while the position of the plunger 978 of the withdrawal syringe 976 remains unchanged with respect to the body 980. As such, when the operating member 986 is moved in the direction 988, screening fluid 14 is dispensed from the syringe 970, but no fluid is withdrawn by the syringe 976. Referring to FIG. 9C, when the operating member 986 of the ratchet mechanism is moved in a direction indicated by arrow 990, the plunger 978 of the withdrawal syringe 976 is pulled outward with respect to the body 980, while the position of the plunger 972 of the screening syringe 970 remains unchanged with respect to the body 974. As such, when the operating member 986 is moved in the direction 990, flush fluid 20 is withdrawn by the withdrawal syringe 976, but no fluid is dispensed by the screening fluid syringe 970. Any ratchet mechanism that moves the plungers 972, 978 in this manner can be used.

In the example illustrated by FIG. 9A, the ratchet mechanism 984 includes the operating member 986, a biasing member 994, one or more screening fluid syringe pawls 996, a screening syringe rack 998, one or more withdrawal syringe pawls 900, and a withdrawal syringe rack 902. The operating member 986 is movable with respect to the frame 982 from the position shown in FIG. 9A to the position shown in FIG. 9B. The biasing member 994 is coupled to the frame 982 and the operating member 986, such that the biasing member 986 returns the operating member to the position shown in FIG. 9A when the operating member is released.

The screening syringe rack 998 and the withdrawal rack 902 include teeth 904. Each tooth 904 includes a steep driven portion 906 and gradually inclined portion 908. In the example illustrated by FIG. 9A, the racks 998, 904 are defined as part of the plungers 972, 978. The racks can be formed in a wide variety of different ways. For example, the racks may be separate members attached to the plungers. The screening fluid syringe pawl(s) 996 and the withdrawal syringe pawl(s) 960 are pivotally connected to the operating member 986 and are positioned in engagement with the screening fluid syringe rack 998 and the withdrawal syringe rack 904.

Referring to FIG. 9B, when the operating member 986 is moved in the direction 988, the screening fluid syringe pawl(s) 996 engage a steep driven portion 906 of a tooth 904 of the screening syringe rack 998 and force the plunger 972 into the body 974. At the same time, the withdrawal syringe pawl(s) 900 slide over the gradually inclined portion 908 of a tooth 904 of the withdrawal rack 904.

Referring to FIG. 9C, when the operating member is released, the biasing member 994 moves the operating member in the direction indicated by arrow 990. The withdrawal syringe pawl(s) 900 engage a steep driven portion 906 of a tooth 904 of the withdrawal rack 904 and force the plunger 978 outward with respect to the body. At the same time, the screening fluid syringe pawl(s) 996 slide over the gradually inclined portion of a tooth 904 of the screening fluid syringe rack 998. The operating member 986 may be moved as indicated by FIG. 9B and allowed to return as indicated by FIG. 9C multiple times to inject and withdraw fluid multiple times. In the example illustrated by FIGS. 9A-9, the operating member 986 is illustrated as causing the pawls to traverse one tooth 904 of the racks at a time. However, the ratchet can be configured such that the pawls can traverse more than one tooth of the rack each time the operating member 986 is moved, depending on how far the operating member is moved. This allows the user to control how much fluid is dispensed and withdrawn by controlling the stroke of the actuating member.

FIG. 10A illustrates another example of a tool 1040. The tool 1040 includes a nozzle 1042 with an outlet port 1048 and an inlet port 1050. The tool 1040 includes a screening fluid supply 1044 and a flush fluid recovery mechanism 1046. In the exemplary tool 1040 illustrated by FIG. 9A, the screening fluid supply 1044 comprises a syringe 1070 that includes a plunger 1072 and a reservoir or body 1074. The flush fluid recovery mechanism 1046 comprises a syringe 1076 that includes a plunger 1078 and a reservoir or body 1080. The syringe bodies 1074, 1080 are fixed to a frame member 1082. The tool 1040 also includes a ratchet mechanism 1084 that is coupled to the screening fluid syringe 1070 and the withdrawal syringe 1076.

Referring to FIG. 10B, when an operating member 1086 of the ratchet mechanism is moved in a direction illustrated by arrow 1088, the plunger 1072 of the fluid screening syringe 1070 is forced into the body 1074, while the position of the plunger 1078 of the withdrawal syringe 1076 remains unchanged with respect to the body 1080. As such, when the operating member 1086 is moved in the direction 1088, screening fluid 14 is dispensed from the syringe 1070, but no fluid is withdrawn by the syringe 1076.

Referring to FIG. 10C, when the operating member 1086 of the ratchet mechanism is moved in a direction indicated by arrow 1090, the plunger 1078 of the withdrawal syringe 1076 is pulled outward with respect to the body 1080, while the position of the plunger 1072 of the screening syringe 1070 remains unchanged with respect to the body 1074. As such, when the operating member 1086 is moved in the direction 1090, flush fluid 20 is withdrawn by the withdrawal syringe 1076, but no fluid is dispensed by the screening fluid syringe 1070.

In the example illustrated by FIG. 10A, the ratchet mechanism 1084 includes the operating member 1086, a biasing member 1094, one or more screening fluid syringe pawls 1096, a screening syringe rack 1098, one or more withdrawal syringe pawls 1000, and a withdrawal syringe rack 1002. In the embodiment illustrated by FIG. 10A, the screening fluid syringe rack 1098 is fixed to the withdrawal syringe rack 1002. The operating member 1086 is movable with respect to the frame 1082 from the position shown in FIG. 10A to the position shown in FIG. 10B. The biasing member 1094 is coupled to the frame 1082 and the operating member 1086, such that the biasing member 1086 returns the operating member to the position shown in FIG. 10A when the operating member is released.

The screening syringe rack 1098 and the withdrawal rack 1002 include teeth 1004. Each tooth 1004 includes a steep drive portion 1006 and gradually inclined portion 1008. In the example illustrated by FIG. 10A, the racks 1098, 1004 are connected to the operating member 1086 and are movable with respect to the Frame between the position shown in FIG. 10A and the position shown in FIG. 10B. The screening fluid syringe pawl 1096 and the withdrawal syringe pawl 900 are coupled to the plungers 1072, 1078 such that the pawls are biased into engagement with the screening syringe rack 998 and the withdrawal syringe rack 904, but are movable away from the racks to facilitate the ratchet action.

Referring to FIG. 10B, when the operating member 1086 is moved in the direction 1088, the steep drive portion 1006 of a tooth 1004 of the screening syringe rack 1098 engages the screening fluid syringe pawl(s) 1096 and forces the plunger 1072 into the body 1074. At the same time, the withdrawal syringe pawl 1000 slides over the gradually inclined portion 1008 of a tooth 1004 of the withdrawal rack 1002.

Referring to FIG. 10C, when the operating member is released, the biasing member 1094 moves the operating member in the direction indicated by arrow 1090. A steep drive portion 1006 of a tooth 1004 of the withdrawal syringe rack engages the withdrawal syringe pawl 1000 and forces the plunger 1078 outward with respect to the body. At the same time, the screening fluid syringe pawls 1096 slide over the gradually inclined portion of a tooth 1004 of the screening fluid syringe rack. The operating member 1086 may be moved as indicated by 10B and be allowed to return as indicated by FIG. 10C multiple times to inject and withdraw fluid multiple times. In the example illustrated by FIGS. 9A-9C, the operating member 986 is illustrated as causing the pawls to transverse one tooth 904 of the racks at a time. In an exemplary embodiment, the ratchet can be configured such that the pawls traverse more than one tooth at a time each time the operating member is moved. The number of teeth traversed depends on how for the operating member is moved and the size of the teeth.

A wide variety of different ratchet mechanisms can be used. In one exemplary embodiment, any arrangement can be used that advances one plunger and retracts another plunger by moving a single operating member or trigger. In the embodiments illustrated by FIGS. 9A and 10A, the tools inject fluid when the operating member or trigger is moved by the user in one direction and withdraw fluid when the operating member or trigger is released. This action can be reversed or the biasing member can be removed such that user action is required both to inject and withdraw fluid. As used herein, the term “ratchet mechanism” is to be broadly interpreted as any mechanism that converts bidirectional motion to unidirectional motion. One ratchet mechanism that may be employed is a wobble plate mechanism that is used in conventional mechanical caulk suns that include smooth shafts rather than shafts with a gear rack defined on the shaft. The ratchet mechanism may be configured such that movement of the operating member in one direction causes the flush fluid plunger and the withdrawal plunger to be alternately advanced and withdrawn during a single stroke of the operating member in one direction. This may be accomplished by employing an escapement mechanism that switches the ratchet mechanism back and forth between an inject state and a withdrawal state during the travel of the operating member.

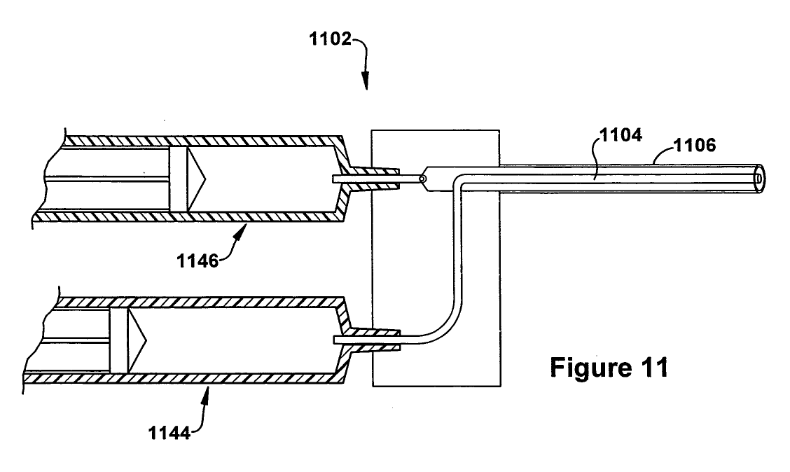

The nozzle of any of the tools can take a wide variety of different forms. Any nozzle configuration that allows the nozzle to be inserted into the body cavity for which the nozzle is designed can be used. FIG. 11 illustrates one nozzle arrangement 1102 that may be used. The nozzle arrangement 1102 includes two substantially coaxial tubes 1104, 1106. The inner tube 1104 is connected to a flush fluid supply 1144 and the outer tube 1106 is connected to a fluid withdrawal mechanism 1146 in the illustrated embodiment, but the connections can be reversed as required by the application.

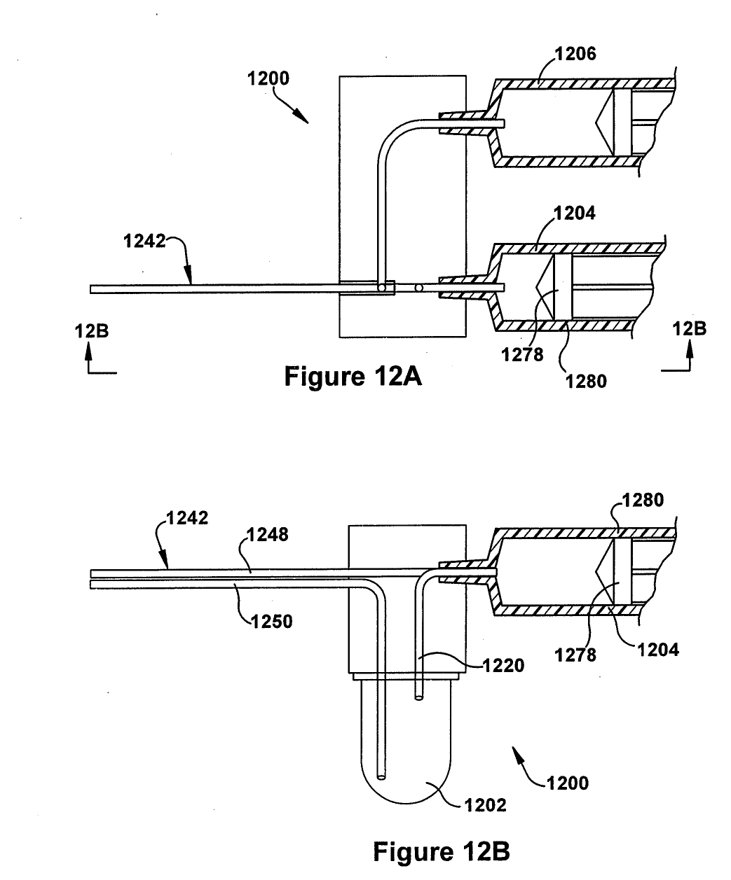

FIGS. 12A and 12B illustrate a flush fluid collection arrangement 1200 where a collection receptacle 1202 is separate from a withdrawal syringe 1204. A supply syringe 1206 is connected to a supply port 1248 of a nozzle 1242. The withdrawal syringe 1204 is in sealed fluid communication with the collection receptacle 1202 via a line 1220. A withdrawal port 1250 of the nozzle 1242 is also in sealed fluid communication with the collection receptacle 1202. When a plunger 1278 of the withdrawal syringe 1204 is withdrawn from the syringe body 1280, a partial vacuum is formed in the collection receptacle 1202, which pulls fluid that the nozzle 1250 is exposed to into the collection receptacle 1202. The use of the collection receptacle 1202 may allow the tool to be used multiple times, without replacing the withdrawal syringe 1204.

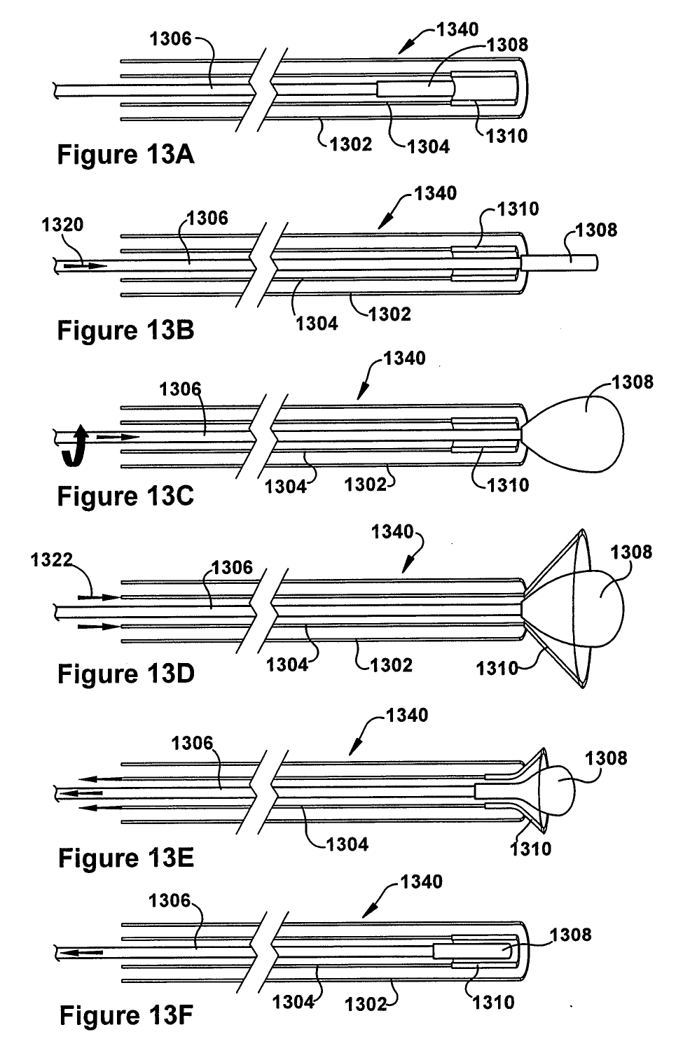

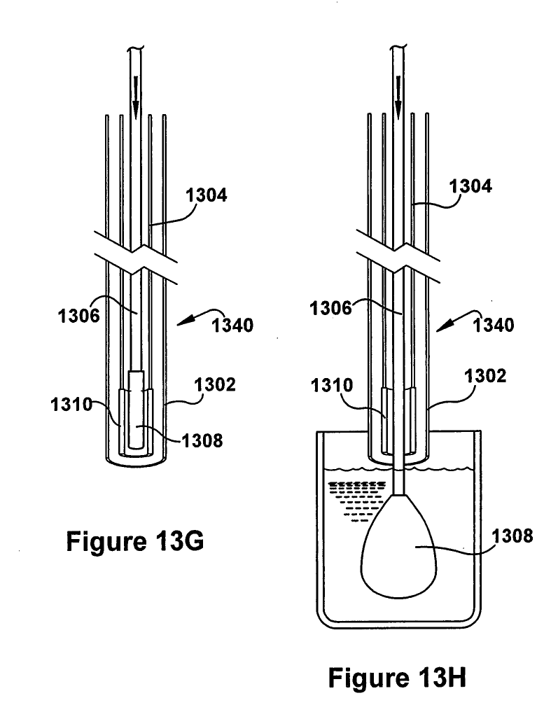

Endometrial cells may be collected in a variety of different ways. FIGS. 13A-13H illustrate another tool 1340 that can be used to collect endometrial cells. FIG. 1340 illustrates the tool 1340 in a stored or retracted mode. The tool 1340 includes an external tube 1302, a collector cover 1304, a collector rod 1306, and a collector head 1308.

The external tube 1302 functions as a handle and houses the collector cover 1304, the collector rod 1306, and the collector head 1308 when the tool 1340 is in the retracted mode. The collector cover 1304 is optional. The collector cover 1304 serves as a sheath for the collector head 1308. A distal end portion 1310 of the collector cover 1304 is configured to expand to a funnel-shaped structure when pushed outside of the external tube 1302 (See FIG. 13D). The collector cover can have any size or shape that may be appropriate for the given application. In one embodiment, the distal end portion 1310 of the collector cover 1304 can be pushed outside the external tube 1302 to a distance of about 1-1.5 centimeters, which allows the distal end portion 1310 to spread like a funnel or cone.

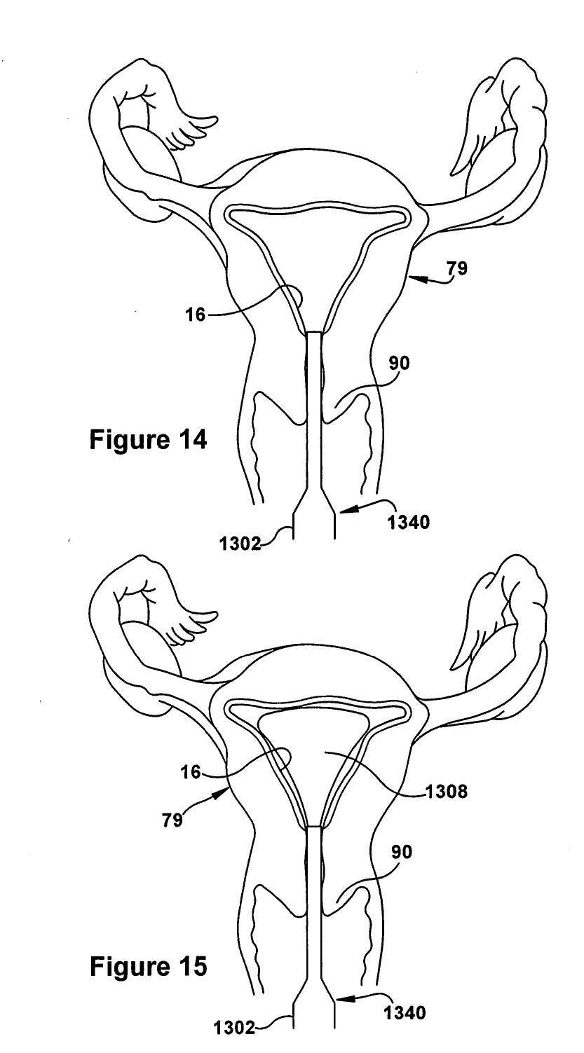

The collector head 1308 is connected to a tip of the collector rod 1306. The collector rod 1306 and the collector head 1308 can be pushed out of or pulled into the external tube 1302 independent of the collector cover 1304. The collector head 1308 is configured to expand and collapse and is made from a material that is suitable for contacting internal human cavity surfaces and collect cells of the cavity wall. The collector head 1308 can be made to expand and contract in a wide variety of different ways. For example, the collector head 1308 may be an expandable bladder or balloon. In the exemplary embodiment, the collector head 1308 and the collector rod 1306 are disposed in the external tube 1302 such that rotating of the collector rod 1306 can cause rotation of the collector head 1308 with respect to the external tube. For example, the collector head may be rotated between forty-five degrees and ninety degrees with respect to the external tube 1302. The rotation of the collector head 1308 causes the collector head to exert a mild shearing force on a cavity wall, such as an endometrium, for better sampling. Referring to FIG. 14, the tool 1340 in the retracted mode may be inserted through a cervix 90, to the uterus 79.

Referring to FIG. 13B, the collector head 1308 is pushed out of the external tube 1302 and the collector cover 1304 by exerting force as indicated by arrow 1320 on the collector rod 1306. The collector head 1308 is now disposed in the uterine cavity in a collapsed state.

Referring to FIGS. 13C and 15, the collector head 1308 is expanded once the collector head 1308 is in the extended position. The collector head can be expanded in a wide variety of different ways. For example, the collector head 1308 can be expanded by injecting a small amount of air through the collector rod 1306. Referring to FIG. 15, the expansion of the collector head 1308 causes the collector head 1308 to come into contact with the surface of the endometrium. The contact between the collector head 1308 and the endometrium cause endometrial cells to become disposed on the collector head.

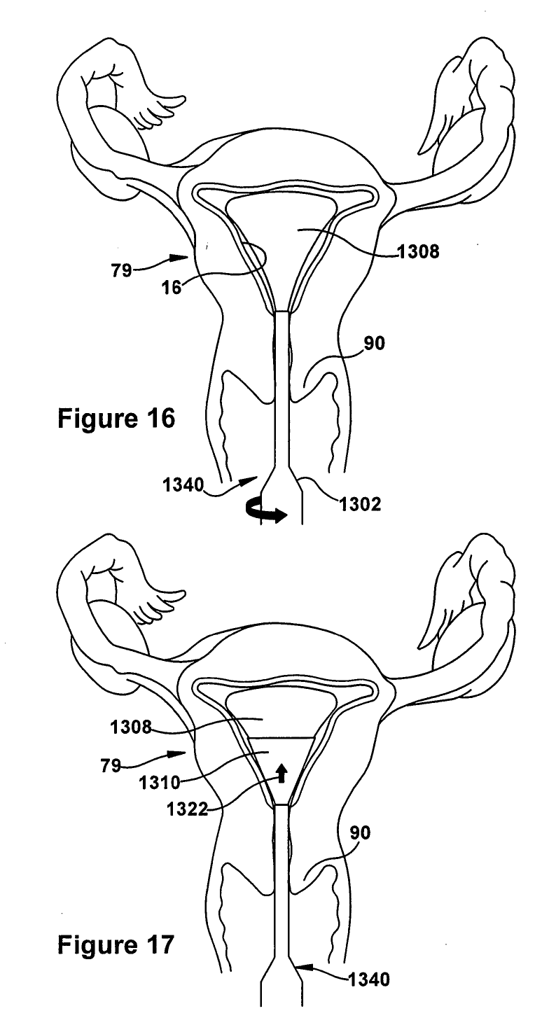

Referring to FIGS. 13C and 16, the collector rod 1306 and therefore, the collector head 1308 may optionally be rotated. This is done manually by the physician, either clock-wise, counter clock-wise, or both. At this stage a representative sample of cells will have been collected from most of the internal (endometrial) surface of the uterus onto the collector head 1308.

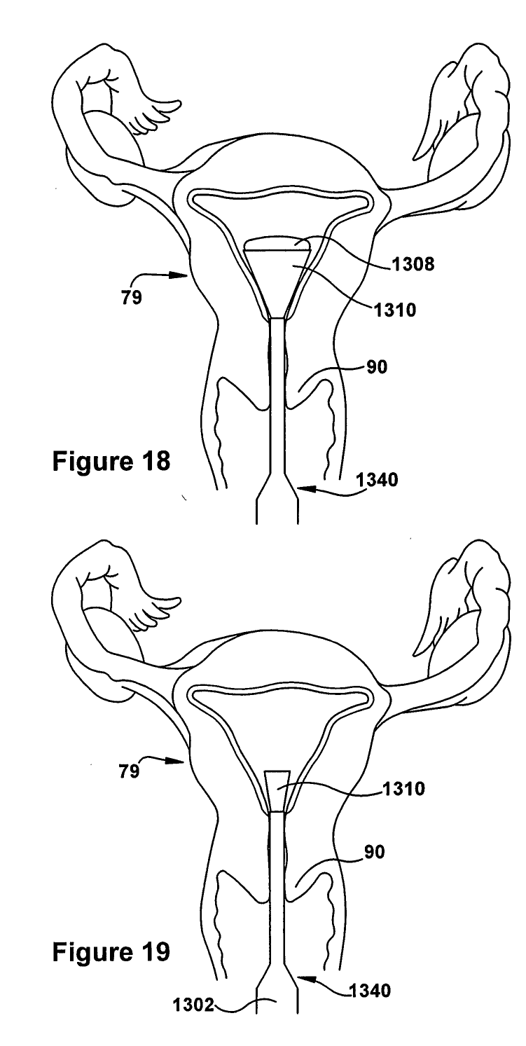

Referring to FIGS. 13D and 17, the distal end portion 1310 of the collector cover 1304 is caused to spread to form a funnel-like configuration by pushing the collector cover in the direction indicated by arrow 1322, i.e., toward the uterus when the tool 1340 is in use. The distal end portion 1310 of the collector cover 1304 may be made from an elastic material to facilitate the funneling action. The degree of funneling will depend mainly on the geometry of the uterine cavity. Referring to FIG. 13D and FIG. 18, the funnel configuration of the distal end portion 1310 acts as a guide for the collector head 1308 as the collector head is collapsed and drawn back into the external tube 1302.

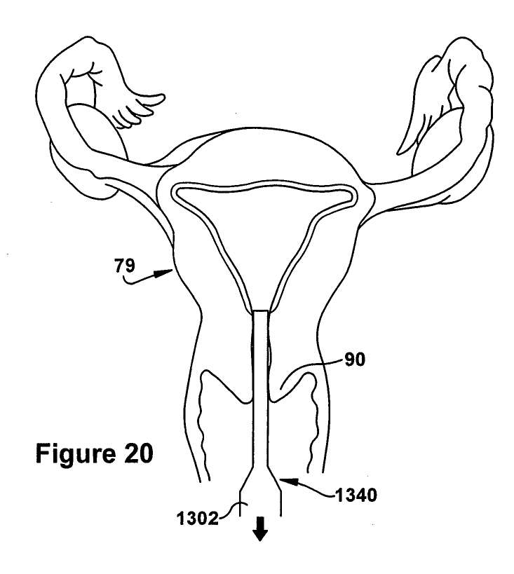

Referring to FIG. 13E, FIG. 18 and FIG. 19, the collector rod 1306 and attached collector head 1308 that is now loaded with endometrial cells and the collector cover 1304 are pulled back into the external tube 1302. The collector head 1308 may be pulled back before the collector cover 1304 is pulled back. Referring to FIGS. 13F and 20, the tool 1340 is now in the withdrawn state and may be pulled out of the uterus and away from the patient.

FIGS. 13F and 13G illustrate the tool 1340 removed from the uterus. The outer tube 1302 containing the collector cover 1304, the collector rod 1306, and the collector head 1308 are moved to a cell removal station. Referring to FIG. 13H, the collector head 1308 is dipped in a cell removal solution 1330 that causes endometrial cells on the collector head to be washed off into the solution. The cell removal solution may take a wide variety of different forms. For example, the cell removal solution may be saline solution. Any solution capable of washing all or a portion of the endometrial cells off of the collector head 1308 may be used. Gentle swirling will release the cells into the solution. At this stage the instrument is thrown away and the cells in the solution are processed to detect one or more indicators of a disease.

While various aspects of the invention are described and illustrated herein as embodied in combination in the exemplary embodiments, these various aspects may be realized in many alternative embodiments, either individually or in various combinations and sub-combinations thereof. Unless expressly excluded herein all such combinations and sub-combinations are intended to be within the scope of the present invention. Still further, while various alternative embodiments as to the various aspects and features of the invention, such as alternative materials, structures, configurations, methods, devices, software, hardware, control logic and so on may be described herein, such descriptions are not intended to be a complete or exhaustive list of available alternative embodiments, whether presently known or later developed. Those skilled in the art may readily adopt one or more of the aspects, concepts or features of the invention into additional embodiments within the scope of the present invention even if such embodiments are not expressly disclosed herein. Additionally, even though some features, concepts or aspects of the invention may be described herein as being a preferred arrangement or method, such description is not intended to suggest that such feature is required or necessary unless expressly so stated. Still further, exemplary or representative values and ranges may be included to assist in understanding the present invention however, such values and ranges are not to be construed in a limiting sense and are intended to be critical values or ranges only if so expressly stated.

While the invention has been described with reference to specific embodiments, it will be apparent to those skilled in the art that may alternatives, modifications, and variations may be made. Accordingly, the present invention is intended to embrace all such alternatives, modifications, and variations that may fall within the spirit and scope of the appended claims.

I claim:

1. A method of screening for endometrial cancer comprising:
providing a flush fluid that is obtained by injecting a screening fluid into an endometrium where epithelial endometrial cells become entrained in the screening fluid to form said flush fluid and withdrawing the flush fluid out of the endometrium;
analyzing the cells from the flush fluid to determine whether the cells exhibit a characteristic that is indicative of the presence of at least one of cancer and a precancerous state.

2. The method of claim 1 further comprising separating the endometrial cells from the fluid before analyzing the cells.

3. The method of claim 1 wherein said characteristic that is indicative of cancer is a condition of at least one biomarker that is indicative of endometrial cancer.

4. The method of claim 3 wherein the at least one biomarker comprises a P2X7 biomarker.

5. The method of claim 1 wherein said characteristic that is indicative of cancer is the presence of a biomarker that is indicative of endometrial cancer.

6. The method of claim 5 wherein the at least one biomarker comprises a P2X7 biomarker.

7. A method of collecting endometrial cells comprising:
injecting a screening fluid into a human uterus;
withdrawing a flush fluid that comprises the screening fluid and entrained endometrial cells from the uterus; and
collecting the withdrawn flush fluid.

8. The method of claim 7 wherein the flush fluid is withdrawn with a vacuum.

9. The method of claim 7 wherein the flush fluid is withdrawn with a nozzle that is disposed in the uterus.

10. The method of claim 7 wherein the flush fluid is injected and withdrawn with a single tool.

11. The method of claim 7 separating the endometrial cells from the fluid.

12. The method of claim 11 further comprising analyzing the endometrial cells from the flush fluid to determine whether a characteristic of the cells is indicative of at least one of cancer in the tissue lining and a pre-cancerous state in the tissue lining.

13. The method of claim 7 wherein the screening fluid is injected into the uterus under pressure.

14. A tool for collecting endometrial cells comprising:
a nozzle configured for insertion into a human uterus, the nozzle including an inlet port and an outlet port;
a screening fluid supply in communication with the outlet port configured to selectively supply a screening fluid through the outlet port and into the uterus;
a vacuum source in communication with the with the inlet port configured to selectively withdraw a flush fluid that includes screening fluid and endometrial cells that are entrained in the screening fluid from the uterus; and
a flush fluid receptacle in communication with the inlet port configured to receive the withdrawn flush fluid.

15. The tool of claim 14 wherein the nozzle is configured to inject the screening fluid and remove flush fluid multiple times without removing the nozzle from the uterus.

16. The tool of claim 14 wherein the vacuum source comprises a first syringe and the screening fluid supply comprises a second syringe and the tool further comprises a single ratchet mechanism that is coupled to the first syringe such that operation of the ratchet mechanism withdraws a plunger of the first syringe from a body of the first syringe and that is coupled to the second syringe such that operation of the ratchet mechanism advances a plunger of the second syringe into a body of the second syringe.

17. The tool of claim 16 wherein the ratchet mechanism is configured to alternately withdraw the plunger of the first syringe and advance the plunger of the second syringe.

18. The tool of claim 17 wherein the ratchet mechanism is configured to provide a time delay between withdrawal of the plunger of the first syringe and advancement of the plunger of the second syringe.

19. The tool of claim 16 wherein the ratchet mechanism includes a single trigger and that actuation of the single trigger causes the ratchet mechanism to alternately withdraw the plunger of the first syringe and advance the plunger of the second syringe.
